(19) United States
(12) Patent Application Publication
(10) Pub. No.: US 2026/0088160 A1
(43) Pub. Date: Mar. 26, 2026

(54) METHODS AND SYSTEMS FOR PREDICTING INTENSIVE CARE UNIT PATIENT LENGTH OF STAY

(71) Applicant: KONINKLIJKE PHILIPS N.V., EINDHOVEN (NL)

(72) Inventors: LOUIS NICOLAS ATALLAH, BOSTON, MA (US); OMAR BADAWI, LUTHERVILLE TIMONIUM, MD (US); XINGGANG LIU, BALTIMORE, MD (US); ROBIN FRENCH, BALTIMORE, MD (US); PAMELA JAYNE AMELUNG JAYNE AMELUNG, BALTIMORE, MD (US)

(21) Appl. No.: 19/109,855

(22) PCT Filed: Sep. 14, 2023
(86) PCT No.: PCT/EP2023/075244

§ 371 Date: Mar. 7, 2025

Related U.S. Application Data
(60) Provisional application No. 63/406,478, filed on Sep. 14, 2022.

(51) Int. Cl. G16H 40/20; G16H 10/60

(52) U.S. Cl. CPC G16H 40/20; G16H 10/60

(57) ABSTRACT

The present disclosure relates to methods and systems for predicting a length of stay (LOS) for a patient in an intensive care unit (ICU). As described herein, the methods and systems for generating a prediction of ICU LOS present improved performance over existing available predictive models. In certain embodiments, the methods described herein include: providing an ICU LOS prediction system; obtaining a plurality of records for a patient in an ICU covering at least a first time period; extracting a plurality of different defined ICU LOS prediction features for the patient; analyzing the extracted plurality of different defined ICU LOS prediction features using a trained developed ICU LOS prediction model; generating a prediction of ICU LOS for the patient based on the analysis; and presenting the generated prediction of ICU LOS for the patient via a user interface.

### FIELD OF THE DISCLOSURE

The present disclosure relates generally to methods and systems for predicting intensive care unit length of stay, and more specifically to methods and systems for predicting intensive care unit length of stay for a patient using risk adjusted models.

### BACKGROUND

Widespread adoption of electronic health records has enabled automated data capturing and propelled predictive risk modeling in a variety of respects and across many different cohorts. In certain settings, risk adjusted predictive modeling has become an essential pillar for measuring outcomes and other benchmarking. For example, a length of stay (LOS) prediction can be used for planning, identifying individuals with unexpectedly long (or even short) LOS, and for benchmarking.

However, the prediction of LOS, and particularly intensive care unit (ICU) LOS is a challenging problem because more severe patients generally have a longer LOS while also having a higher risk of death, which would decrease their LOS. Further, LOS distributions are asymmetrical and present multimodality due to, in part, patient discharge happening at preferred times during the day. As a result, existing methods and systems for predicting a LOS for an ICU patient fail to capture these and other aspects of this complex relationship.

### SUMMARY OF THE DISCLOSURE

Accordingly, there is a continued need for improved prediction of a patient's length of stay in an ICU.

According to an embodiment of the present disclosure, a method for predicting length of stay (LOS) for a patient in an intensive care unit (ICU) using an ICU LOS prediction system is provided. The method comprises: providing (110) an ICU LOS prediction system (200); obtaining (120), from an electronic medical records database (270), a plurality of records for a patient in an ICU covering at least a first time period; extracting (130), from the obtained plurality of records, a plurality of different defined ICU LOS prediction features for the patient, wherein at least some of the plurality of different defined ICU LOS prediction features are required ICU LOS prediction features and wherein at least some of the plurality of different defined ICU LOS prediction features are not required ICU LOS prediction features; analyzing (140) the extracted plurality of different defined ICU LOS prediction features using a trained ICU LOS prediction model (264); generating (150), from the analysis, a prediction of ICU LOS for the patient, wherein the ICU LOS prediction comprises a predicted LOS for the patient; presenting (160), via a user interface (240) of the ICU LOS prediction system, the generated prediction of ICU LOS for the patient. The ICU LOS prediction model may be trained, validated and tested by: (i) obtaining (310), from an electronic medical records database (270), a plurality of records for each of a plurality of patients in an ICU covering at least a first time period; (ii) extracting (320), from the obtained plurality of records, a plurality of different health features for each of the plurality of patients; (iii) manually curating (330), based on the results of extracting, the extracted plurality of different health features to identify the plurality of different defined ICU LOS prediction features, wherein manually curating comprise identifying which of the different health features are the required ICU LOS prediction features and which of the different health features are not required ICU LOS prediction features; (iv) training (340) the ICU LOS prediction model using the plurality of different defined ICU LOS prediction features for at least some of the plurality of patients; and (v) storing (350) the trained ICU LOS prediction model.

In an aspect, the first time period of data acquisition is at least 24 hours.

In an aspect, the extracted plurality of different defined ICU LOS prediction features for the patient comprises some or all of the features in TABLE 1.

In an aspect, the ICU LOS prediction system is, or is a component of, a patient data management systems (PDMS), a patient monitoring system, a patient flow management system or an ICU retrospective analysis system.

In an aspect, the patient is a historical patient.

In an aspect, the trained ICU LOS prediction model can analyze the extracted plurality of different defined ICU LOS prediction features and generate a prediction of ICU LOS for the patient when some or all of the not required ICU LOS prediction features are missing from the obtained plurality of records.

In an aspect, the generated prediction of ICU LOS for the patient comprises an estimated probability of the patient leaving the ICU conditioned on a patient discharge status at a plurality of different timepoints or the expected time of discharge calculated based on such probabilities.

In an aspect, at least some of the extracted plurality of different health features are binned prior to confection of inputs to the ICU LOS prediction model.

In an aspect, the ICU LOS prediction model comprises time-to-event analysis with competing risks, the time-to-event comprising time elapsed from the patient's admission to the ICU until a time the patient leaves the ICU, and the competing risks comprising an exit status of the patient either alive or deceased.

According to another embodiment of the present disclosure, an intensive care unit (ICU) length of stay (LOS) prediction system (200) configured to predict an LOS for a patient in the ICU is provided. The ICU LOS prediction system comprises: an electronic medical records database (270) comprising a plurality of records for a plurality of patients; a trained ICU LOS prediction model (264) configured to analyze a plurality of different defined ICU LOS prediction features to generate a prediction of ICU LOS for the patient, wherein the ICU LOS prediction comprises a predicted LOS for the patient; a processor (220) configured to: (i) obtain, from the electronic medical records database, a plurality of records for a patient in an ICU covering at least a first time period; (ii) extract, from the obtained plurality of records, a plurality of different defined ICU LOS prediction features for the patient; (iii) analyze the extracted plurality of different defined ICU LOS prediction features using the trained ICU LOS prediction model; and (iv) generate, from the analysis, a prediction of ICU LOS for the patient; and a user interface (240) configured to provide the generated prediction of ICU LOS for the patient. The ICU LOS prediction model may be trained by: (i) obtaining (310), from an electronic medical records database (270), a plurality of records for each of a plurality of patients in an ICU covering at least a first time period; (ii) extracting (320), from the obtained plurality of records, a plurality of different health, demographic and patient characteristic features for each of the plurality of patients; (iii) manually curating (330), based on the results of extracting, the extracted plurality of different health features to identify the plurality of different defined ICU LOS prediction features, wherein manually curating comprise identifying which of the different health features are the required ICU LOS prediction features and which of the different health features are not required ICU LOS prediction features; (iv) training (340) the ICU LOS prediction model using the plurality of different defined ICU LOS prediction features for at least some of the plurality of patients; and (v) storing (350) the trained ICU LOS prediction model.

In an aspect, the extracted plurality of different defined ICU LOS prediction features for the patient comprises some or all of the features in TABLE 1.

In an aspect, the first time period is at least 24 hours.

In an aspect, the ICU LOS prediction system is, or is a component of, a patient data management systems (PDMS) or a patient monitoring system.

In an aspect, the generated prediction of ICU LOS for the patient comprises an estimated probability of the patient leaving the ICU conditioned on a patient discharge status at a plurality of different timepoints.

In an aspect, ICU LOS prediction model comprises time-to-event analysis with competing risks, the time-to-event comprising time elapsed from the patient's admission to the ICU until a time the patient leaves the ICU, and the competing risks comprising an exit status of the patient either alive or deceased.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for predicting a length of stay (LOS) for a patient in an intensive care unit (ICU), illustrated according to aspects of the present disclosure.

FIG. 2 is a schematic diagram of an ICU LOS prediction system, illustrated according to aspects of the present disclosure.

FIG. 3 is a flowchart of a method for training an ICU LOS prediction model, illustrated according to aspects of the present disclosure.

FIG. 4A is a graph of the actual to predicted (A:P) ratio for an inventive example and a comparative example, illustrated according to aspects of the present disclosure.

FIG. 4B is a graph of the MedAE value for an inventive example and a comparative example, illustrated according to aspects of the present disclosure.

FIG. 4C is a graph of the R² value for an inventive example and a comparative example, illustrated according to aspects of the present disclosure.

FIG. 5 is a graph of the distribution of A:P ratios across different LOS predictions, illustrated according to aspects of the present disclosure.

FIG. 6A is a graph of the actual to predicted (A:P) ratio for another inventive example and a comparative example, illustrated according to aspects of the present disclosure.

FIG. 6B is a graph of the MedAE value for another inventive example and a comparative example, illustrated according to aspects of the present disclosure.

FIG. 6C is a graph of the R² value for another inventive example and a comparative example, illustrated according to aspects of the present disclosure.

FIG. 7 is another graph of the distribution of A:P ratios across different LOS predictions, illustrated according to aspects of the present disclosure.

### DETAILED DESCRIPTION OF EMBODIMENTS

The present disclosure is directed to methods and systems for predicting a length of stay (LOS) for a patient in an intensive care unit (ICU). As described herein, the methods and systems use a deep learning model and a plurality of prediction features from information available in the ICU during a first period of time of the patient's stay to generate a predicted LOS for the patient. In embodiments, the methods and systems described enable LOS predictions that account for the complex relationship between a patient's LOS and the plurality of predictive features, and that achieve improved performance for both surviving and non-surviving patients.

The embodiments and implementations disclosed or otherwise envisioned herein can be utilized with any patient care system, including but not limited to clinical decision support tools, benchmarking tools, among other systems. For example, one application of the embodiments and implementations herein is to improve analysis systems such as, e.g., the Philips® eCareManager Enterprise telehealth products, Philips® Tasy EMR solutions, and Philips® Patient Flow Capacity Suite products, among many others. However, the disclosure is not limited to these devices or systems, and thus disclosure and embodiments disclosed herein can encompass any device or system capable of generated and reporting information about the length of stay of a patient.

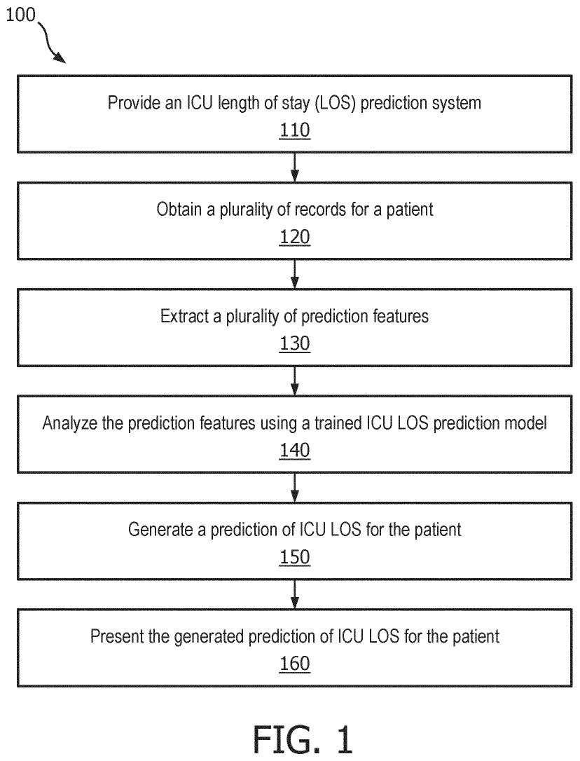

Turning to FIG. 1, a flowchart of a method 100 for predicting a LOS for a patient in an ICU using an ICU LOS prediction system is illustrated according to aspects of the present disclosure.

At a step 110 of the method 100, according to an embodiment, an ICU LOS prediction system 200 is provided. As discussed in greater detail below, the ICU LOS prediction system 200 can be configured predict a LOS for a patient in an ICU. In embodiments, the ICU LOS prediction system 200 can include a trained ICU LOS prediction model 264 configured to analyze a plurality of different defined ICU LOS prediction features to generate a prediction of ICU LOS for the patient. In some embodiments, one or more ICU LOS prediction model(s) 264 may be developed using a deep learning method, such as the DeepHit method, to model the time-to-event with competing risks. As used herein, the term “time-to-event” refers to the time elapsed from ICU admission until the moment the patient leaves the ICU for that stay, while the term “event” refers to the exit status of the patient (i.e., alive or deceased).

At a step 120, the method 100 includes obtaining a plurality of records for a patient in an ICU. In some embodiments, the plurality of records for the patient may be obtained by the ICU LOS prediction system 200. In further embodiments, the plurality of records for the patient may be obtained from an electronic medical records database 270A, 270B. For example, the electronic medical records database 270A, 270B may include patient unit stays admitted to ICUs where physiologic, diagnosis, and treatment information (collectively, “medical information” or “medical data”) are captured, charted, or otherwise recorded. That is, the electronic medical records database 270A, 270B can comprise a plurality of healthcare-related records for a plurality of patients, including historical patients and/or patients of current ICU stays.

In still further embodiments, the plurality of records obtained in step 120 can include medical records that cover a first period of time. For example, the plurality of records obtained in step 120 can include medical records that cover the first 24 hours of the patient's stay in an ICU (i.e., the medical data available through the first day of ICU admission), although longer and shorter periods of time are possible.

Alternatively, if medical records for the patient within the first day of ICU admission are not available, the medical records may include, for example and without limitation, medical records covering up to six hours before ICU admission, although longer and shorter time periods are possible.

As such, in various examples, the first period of time can include the first 24 hours of the patient's ICU stay, only the first 24 hours of the patient's ICU stay, less than 24 hours of the patient's ICU stay, an amount of time (e.g., 1 hour, 3 hours, 6 hours, 12 hours, etc.) preceding the patient's admission to the ICU, and/or some combination thereof. The time period considered for model input acquisition can subsequently be comprised of a shifting time window of fixed length, that allows for the model to produce a prediction based on the fixed amount of time prior to the moment of the production of a prediction.

At a step 130, the method 100 includes extracting a plurality of different defined ICU LOS prediction features for the patient from the plurality of records obtained in step 120. In embodiments, the ICU LOS prediction system 200 may extract the plurality of different defined ICU LOS prediction features for the patient based on the plurality of records obtained in step 120.

As used herein, the term “defined ICU LOS prediction features” refers to physiologic, diagnosis, and/or treatment information that are defined prior to analyzing the plurality of medical records of the patient using a trained model. In embodiments, the plurality of different defined ICU LOS prediction features can include of the defined prediction features shown in Table 1 below:

TABLE 1
ICU LOS PREDICTION FEATURES

| Variables/top categories | Required? | Units |
|---|---|---|
| ICU length of stay | Yes | mean (SD) in hours |
| ICU discharge status deceased | Yes | total count (%) |
| Age | Yes | mean (SD) in years |
| Sex: non-female | Yes | total count (%) |
| Pre ICU admission lead time | Yes | mean (SD) in hours |
| Elective surgery: yes/no | Yes | total count (%) |
| Ventilated: yes/no | Yes | total count (%) |
| Artificial airway: yes/no | Yes | total count (%) |
| BMI | Yes | mean (SD) in kg/m2 |
| Mean blood pressure | Yes | mean (SD) in mmHg |
| Diastolic blood pressure | Yes | mean (SD) in mmHg |
| Systolic blood pressure | Yes | mean (SD) in mmHg |
| Heart rate | Yes | mean (SD) bpm |
| Oxygen saturation | Yes | mean (SD) % |
| Respiratory rate | Yes | mean (SD) bpm |
| PaCO₂ | No | mean (SD) mmHg |
| Glucose | Yes | mean (SD) mg/dL |
| Lactate | No | mean (SD) mmol/L |
| pH | No | mean (SD) |
| White blood cell count | Yes | mean (SD) count per ml |
| Hemoglobin | Yes | mean (SD) g/dL |
| Albumin | No | mean (SD) g/dL |
| Sodium | Yes | mean (SD) mEq/L |
| Potassium | Yes | mean (SD) mEq/L |
| Creatinine | Yes | mean (SD) mmol/L |
| GCS total | No | total count (%) |
| ICU type | Yes | total count (%) |
| ICU admission source | Yes | total count (%) |
| Diagnostic group | Yes | total count (%) |

As shown in Table 1, the plurality of different defined ICU LOS prediction features can include one or more different variables. In some embodiments, one or more of these ICU LOS prediction features may be required, while others may not be required, as shown in Table 1. In particular embodiments, the ICU LOS prediction features include only those features identified in Table 1. In other embodiments, the plurality of different defined ICU LOS prediction features may not be limited to only these features, and it is contemplated that other variables may be included in future models.

In embodiments, the plurality of different defined ICU LOS prediction features may be automatically extracted from the plurality of records obtained in step 120 using natural language processing and/or a machine learning algorithm. For example, the ICU LOS prediction system 200 may include a prediction feature extractor 261 that implements a natural language processing technique and/or a machine learning algorithm in order to extract the plurality of different defined ICU LOS prediction features.

At a step 140, the method 100 includes analyzing the extracted plurality of different defined ICU LOS prediction features using a trained ICU LOS prediction model 264. In embodiments, the ICU LOS prediction system 200 can apply the trained ICU LOS prediction model(s) 264 to the extracted plurality of different defined ICU LOS prediction features.

In embodiments, the extracted plurality of different defined ICU LOS prediction features may be analyzed by feeding one or more of the different defined ICU LOS prediction features into the trained ICU LOS prediction model 264, which may be, for example, a deep neural network model. In some embodiments, the trained ICU LOS prediction model 264 may be a DeepHit model that has the ability to handle competing risks in which there are mutually exclusive events of interest, such as a patient discharge disposition (e.g., surviving/non-surviving). In specific embodiments, the trained ICU LOS prediction model 264 may comprise a multitask network of two cause-specific networks and one shared subnetwork with a SoftMax output layer.

As an output, the trained ICU LOS prediction model 264 may produce an array of estimated probabilities of the patient leaving the ICU either alive or deceased at one or more hours in the future. For example, the SoftMax layer of the trained ICU LOS prediction model 264 may output an array q (2×720), where the j-th element of the i-th component, q(i,j), corresponds to the estimated probability that the patient will leave the ICU in a state i (0 for alive, 1 for deceased) in the j-th hour.

At a step 150, the method 100 includes generating a prediction of ICU LOS for the patient based on the analysis performed in step 140. In embodiments, the generated prediction includes a predicted length of stay for the patient that is conditioned on a discharge status of the patient. For example, the patient's discharge status may be living or may be deceased. In embodiments, the generated prediction can include a probability for a plurality of lengths of stay for the patient conditioned on the patient's discharge status. In other words, the generated prediction may comprise an estimated probability of the patient leaving the ICU conditioned on a patient discharge status at a plurality of different timepoints.

For example, in particular embodiments, the output array q determined by the trained ICU LOS prediction model 264 may be used to calculate the patient LOS in one or more ways. In some embodiments, an ICU LOS prediction may be generated with knowledge/awareness of a discharge state for the patient. For example, for a discharge state i, the patient expected LOS (Li) conditioned to the exit status i estimated by the model for the patient leaving the ICU may be determined according to Equation 1:

$${\cdot }_{\cdot } = \frac{\cdot \dot{…} \cdot \cdot ( \cdot , \cdot )}{\cdot \dot{…} \cdot ( \cdot , \cdot )} \qquad ( Equation 1 )$$

In further embodiments, an ICU LOS prediction may be generated that is agnostic as to the outcome (i.e., without considering any knowledge of the final ICU exit state). For example, the model predicted expected LOS (L) may be determined according to Equation 2:

$$\cdot = \cdot .. \{ . , . \} \cdot \dot{…} \cdot \cdot ( \cdot , \cdot ) \qquad ( Equation 2 )$$

where i=0 if the patient left the ICU alive, or i=1 if they left the ICU deceased.

In still further embodiments, the estimated probability of a patient to be discharged before n hours may be determined according to Equation 3:

$$\cdot .. \{ . , . \} {\cdot }_{\cdot \cdot \cdot }^{\cdot \cdot \cdot } \cdot ( \cdot , \cdot ) \qquad ( Equation 3 )$$

In some embodiments, the predicted LOS values may then be compared to a true LOS by means of the coefficient of determination (R²), concordance index (C-index), and/or the median absolute error. The R² metric indicates how much of the variability of true LOS is explained by the predicted LOS, where the best possible value is one. The concordance index is a measure of rank correlation between the predicted probabilities and observed durations of stay ranging from 0 to 1 (with a perfect pairwise concordance being equal to 1 while a random pairing being equal to 0.5).

In embodiments, one or more of these metrics may be calculated at the level of a single stay for a patient and/or at the ICU level for multiple patients. In particular embodiments, the prediction of ICU LOS for a patient can include a predicted LOS for the patient conditioned on discharge status (i.e., alive or deceased).

At a step 160, the method 100 includes presenting the generated prediction of ICU LOS for the patient. For example, in embodiments, the generated prediction of ICU LOS for the patient may be presented to a healthcare worker, administrator, and/or provider responsible for the patient. In some embodiments, the generated prediction of ICU LOS for the patient may be presented via a user interface, such as a display screen or computer monitor. In embodiments, the user interface used to present the generated prediction of ICU LOS for the patient may be a user interface 240 of the ICU LOS prediction system 200. In still further embodiments, the patient is still admitted to the ICU while the prediction of ICU LOS is generated and/or presented (e.g., the method 100 is performed before the patient is discharged from the ICU).

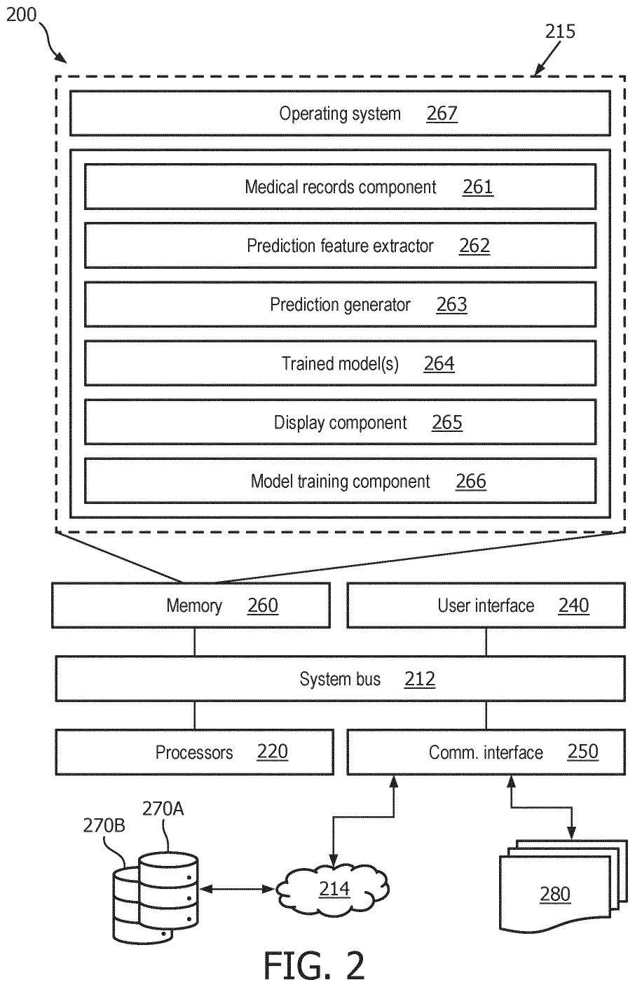

Turning to FIG. 2, an example ICU LOS prediction system 200 is illustrated. The ICU LOS prediction system 200 can be configured to generate a prediction of ICU LOS for a patient, as described above. In some embodiments, the ICU LOS prediction system 200 may be at least part of a larger patient data management system (PDMS), a patient monitoring system, a patient flow management system, and/or an ICU reporting and benchmarking system.

In embodiments, the ICU LOS prediction system 200 comprises one or more processors 220, machine-readable memory 260, a user interface 240, and/or a communications interface 250, all of which may be interconnected and/or communication through a system bus 212 containing conductive circuit pathways through which instructions (e.g., machine-readable signals) may travel to effectuate communication, tasks, storage, and the like.

As discussed in more detail below, the one or more processors 220 may be configured to perform one or more steps of the methods described herein, including but not limited to, the following: (i) obtain, from an electronic medical records database 270A, 270B, a plurality of records for one or more patients in an ICU covering at least a first time period; (ii) extract, from the obtained plurality of records, a plurality of different defined ICU LOS prediction features for one or more patients; (iii) analyze the extracted plurality of different defined ICU LOS prediction features using a trained ICU LOS prediction model 264; and (iv) generate, from the analysis, a prediction of ICU LOS for one or more patients.

In some examples, the one or more processors 220 may include a high-speed data processor adequate to execute the program components described herein and/or various specialized processing units as may be known in the art. In some examples, the one or more processors 220 may be a single processor, multiple processors, or multiple processor cores on a single die.

In some examples, the communications interface 250 can include a network interface configured to connect the ICU LOS prediction system 200 to a communications network 214, an input/output (“I/O”) interface configured to connect and communicate with one or more peripheral devices, a memory interface configured to accept, communication, and/or connect to a number of machine-readable memory devices, and the like.

In certain embodiments, the communications interface 250 may operatively connect the ICU LOS prediction system 200 to a communications network 214, which can include a direct interconnection, the Internet, a local area network (“LAN”), a metropolitan area network (“MAN”), a wide area network (“WAN”), a wired or Ethernet connection, a wireless connection, and similar types of communications networks, including combinations thereof. In some examples, ICU LOS prediction system 200 may communicate with one or more remote/cloud-based servers (e.g., the electronic medical records database 270A), cloud-based services, and/or remote devices via the communications network 214.

The memory 260 can be variously embodied in one or more forms of machine-accessible and machine-readable memory. In some examples, the memory 260 includes a storage device that comprises one or more types of memory. For example, a storage device can include, but is not limited to, a non-transitory storage medium, a magnetic disk storage, an optical disk storage, an array of storage devices, a solid-state memory device, and the like, including combinations thereof.

Generally, the memory 260 is configured to store data/information and instructions 215 that, when executed by the one or more processors 220, causes the ICU LOS prediction system 200 to perform one or more tasks. In particular examples, the memory 260 includes an ICU LOS prediction package 230 that causes the ICU LOS prediction system 200 to perform one or more steps of the methods described herein.

In embodiments, the ICU LOS prediction package 230 comprises a collection of program components, database components, and/or data. Depending on the particular implementation, the ICU LOS prediction package 230 may include software components, hardware components, and/or some combination of both hardware and software components.

The ICU LOS prediction package 230 may include one or more software packages configured to generate a prediction of ICU LOS for a patient. These software packages may be incorporated into, loaded from, loaded onto, or otherwise operatively available to and from the ICU LOS prediction system 200.

In some examples, the ICU LOS prediction package 230 and/or one or more individual software packages may be stored in a local storage device 260. In other examples, the ICU LOS prediction package 230 and/or one or more individual software packages may be loaded onto and/or updated from a remote server via the communications interface 250.

In particular embodiments, the ICU LOS prediction package 230 can include, but is not limited to, instructions 215 having a medical records component 261, prediction feature extractor 262, a prediction generator 263, one or more trained ICU LOS prediction models 264, a display component 263, and/or a model training component 266. These components may be incorporated into, loaded from, loaded onto, or otherwise operatively available to and from the ICU LOS prediction system 200.

In embodiments, the medical records component 260 can be a stored program component that is executed by at least one processor, such as the one or more processors 220 of the ICU LOS prediction system 200. In particular, the medical records component 260 can be configured to interface with an electronic medical records database 270A in order to obtain a plurality of records for one or more patients, as described herein. That is, the medical records component 260 may be configured to request, receive, and/or otherwise obtain a plurality of medical records for one or more patients in an ICU.

In embodiments, one or more of the patients may be historical patients. In other embodiments, one or more of the patients may be current ICU patients. In still further embodiments, the medical records component 260 may obtain a plurality of records for a combination of historical and/or current ICU patients.

In embodiments, the prediction feature extractor 261 can be a stored program component that is executed by at least one processor, such as the one or more processors 220 of the ICU LOS prediction system 200. In particular, the prediction extractor 261 can be configured to extract a plurality of different defined ICU LOS prediction features for a patient, as described herein. In particular, the prediction feature extractor 261 can be configured to extract defined ICU LOS prediction features from the plurality of records obtained from an electronic medical records database 270A using natural language processing and/or a machine learning algorithm.

In embodiments, the prediction generator 263 can be a stored program component that is executed by at least one processor, such as the one or more processors 220 of the ICU LOS prediction system 200. In particular, the prediction generator 263 can be configured to analyze the extracted plurality of different defined ICU LOS prediction features and generate a prediction of ICU LOS, as described herein.

In particular embodiments, the prediction generator 263 can be configured to use one or more trained ICU LOS prediction model(s) 264 in order to analyze the extracted ICU LOS prediction features. Based on the output of applying the one or more trained ICU LOS prediction model(s) 264, the prediction generator 263 may generate a prediction of ICU LOS for a particular patient.

In embodiments, the display component 265 can be a stored program component that is executed by at least one processor, such as the one or more processors 220 of the ICU LOS prediction system 200. In particular, the display component 265 can be configured operate a user interface 240 in order to present the generated prediction of ICU LOS for the patient, as described herein. In some embodiments, the display component 265 can include a programmable processor, also referred to as a graphics progressing units (GPU), which is specialized for rendering images on a monitor or display screen of a user interface 240. In other words, the user interface 240 may be configured, via a display component 265, to provide or otherwise present a prediction of ICU LOS generated for one or more patients.

The ICU LOS prediction system 200 may also include an operating system component 267, which may be stored in the memory 260. The operating system component 267 may be an executable program facilitating the operation of the ICU LOS prediction system 200. Typically, the operating system component 267 can facilitate access of the communications interface 250, and can communicate with other components of the ICU LOS prediction system 200, including but not limited to, the user interface 240, the memory 260, and/or the electronic medical records database 270A.

According to certain embodiments, the ICU LOS prediction system 200 includes at least an electronic medical records database 270A, 270B, a processor 220, a user interface 240, and a trained ICU LOS prediction model 264. In embodiments, the ICU LOS prediction model 264 may be trained by a training component 266 using a training dataset 280 comprising a plurality of records for each of a plurality of patients over a period of time covering each patient's stay in an ICU.

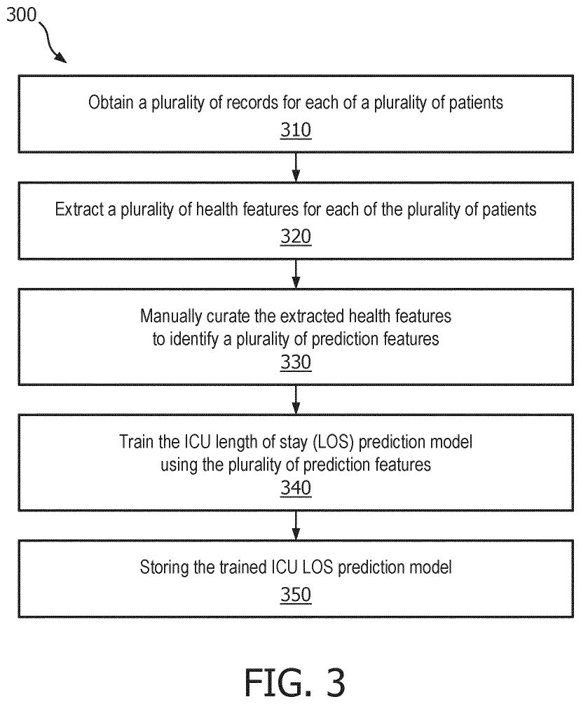

For example, with reference to FIG. 3, a flowchart of a method 300 for training an ICU LOS prediction model is illustrated according to aspects of the present disclosure. In embodiments, the ICU LOS prediction model may be trained by the ICU LOS prediction system 200 and/or may be provided to the ICU LOS prediction system 200 after having already been trained by another similar system.

At a step 310, the method 300 includes obtaining a training dataset 280 comprising a plurality of records for a plurality of patients. In embodiments, the plurality of records for the plurality of patients may be obtained from an electronic medical records database 270B. For example, the electronic medical records database 270B may include patient unit stays admitted to ICUs where physiologic, diagnosis, demographic and treatment information (collectively, “medical information” or “medical data”) are captured, charted, or otherwise recorded. In embodiments, this may be the same electronic medical records database 270A, or may be a different electronic medical records database 270B. In embodiments, the use of the electronic medical records database 270 may be certified as necessary regulatory and privacy standards.

In embodiments, the plurality of records obtained in step 310 can include medical records that cover at least a first period of time for each of the plurality of patients. For example, the plurality of records obtained in step 310 can include medical records that cover the first 24 hours of each patients' stay in an ICU (i.e., the medical data available through the first day of ICU admission). Alternatively, if medical records for one or more of the patients within the first day of ICU admission are not available, the medical records may include, for example and without limitation, medical records covering up to six hours before ICU admission. Many other time periods are possible.

As such, in various examples, the first period of time covered by each of the plurality of medical records can include the first 24 hours of a patient's ICU stay, only the first 24 hours of a patient's ICU stay, less than 24 hours of a patient's ICU stay, an amount of time (e.g., 1 hour, 3 hours, 6 hours, 12 hours, etc.) preceding a patient's admission to the ICU, and/or some combination thereof.

At a step 320, the method 300 includes extracting a plurality of health features for each of the plurality of patients from the training dataset 280 obtained in step 310. In embodiments, these health features may be clinical features representing a patient's ICU stay. For example, continuous features commonly measured (e.g., vital signs, chemistry labs, basic characteristics, etc.) may be included, as well as other continuous features less commonly measured (e.g., lactate, pH, etc.).

At a step 330, the method 300 includes curating the extracted plurality of health features in order to identify and define a set of ICU LOS prediction features. That is, the extracted plurality of health features may be curated to identify and define the plurality of different defined ICU LOS prediction features (such as the plurality of defined ICU LOS prediction features used in steps 130, 140 of a method 100). In particular embodiments, the step 330 can include manually curating one or more of the plurality of different health features extracted in step 320.

At a step 340, the method 300 includes training the ICU LOS prediction model using the plurality of different defined ICU LOS prediction features curated in step 330. In embodiments, the ICU LOS prediction model may be trained using a plurality of different defined ICU LOS prediction features corresponding to at least some of the plurality of patients for which medical records were obtained in step 310 (i.e., the training dataset 280).

At a step 350, the method 300 includes storing the trained ICU LOS prediction model 264. In embodiments, the trained ICU LOS prediction model 264 may be stored in the memory 260 of an ICU LOS prediction system 200. In other embodiments, the trained ICU LOS prediction model 264 may be stored remotely from an ICU LOS prediction system 200, such as in a remote database accessible by an ICU LOS prediction system 200 (e.g., via communications interface 250 and network 214).

As described herein, the methods and systems of predicting a prediction of ICU LOS for a patient achieve improved performance over existing approaches. For example, a comparison of an inventive method and system were compared with an existing model, APACHE IVb, the results of which are shown in Tables 2.1 and 2.2:

TABLE 2.1
PERFORMANCE METRICS FOR
INVENTIVE MODEL EXAMPLE

| | | | Concordance | Median Absolute |
|---|---|---|---|---|
| Level | Population | R² | Index | Error (hours) |
| Individual | All | 0.3 | 0.7 | 25.5 |
| Stays | Surviving | 0.3 | 0.7 | 24.8 |
| | Non-surviving | 0.23 | 0.72 | 51.4 |
| Aggregated | All | 0.48 | 0.75 | 9.1 |
| by ICUs | Surviving | 0.45 | 0.73 | 8.7 |
| | Non-surviving | 0.23 | 0.68 | 22.5 |

TABLE 2.2
PERFORMANCE METRICS FOR APACHE IVB

| | | | Concordance | Median Absolute |
|---|---|---|---|---|
| Level | Population | R² | Index | Error (hours) |
| Individual | All | 0.09 | 0.63 | 32.1 |
| Stays | Surviving | 0.11 | 0.64 | 30.5 |
| | Non-surviving | 0 | 0.52 | 74.1 |
| Aggregated | All | 0.26 | 0.67 | 12.1 |
| by ICUs | Surviving | 0.22 | 0.66 | 11.6 |
| | Non-surviving | 0 | 0.55 | 28.7 |

As shown in Tables 2.1 and 2.2, the inventive ICU LOS model presented a coefficient of determination equal to 0.30 for the entire population and surviving cohorts and 0.23 for the non-surviving population, while APACHE IVb presented a coefficient of determination equal to 0.11 for the surviving population and did not present a positive R² value for the non-surviving population. At the aggregate ICU level, the inventive ICU LOS model presented an R² value equal to 0.48 for all patients (and 0.45 for surviving and 0.23 for non-surviving) compared to APACHE IVb that presented an R² value equal to 0.26 for all patients (and 0.22 for surviving and 0 for non-surviving patients).

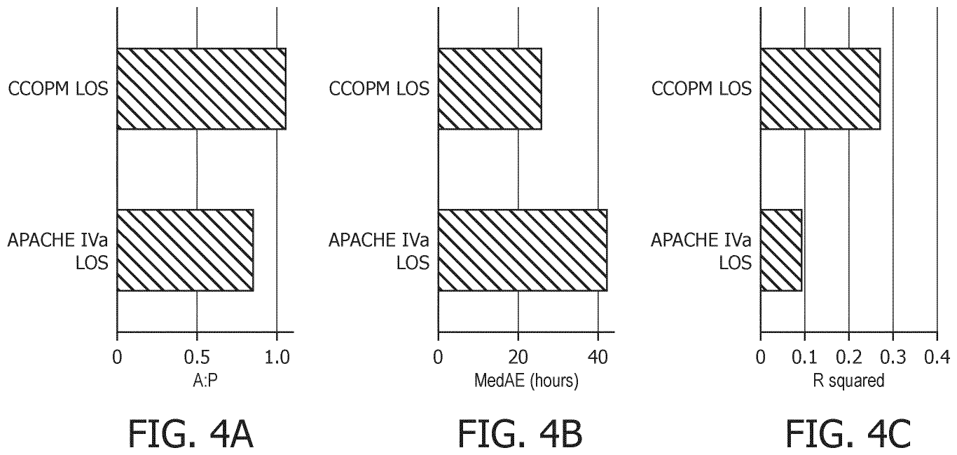

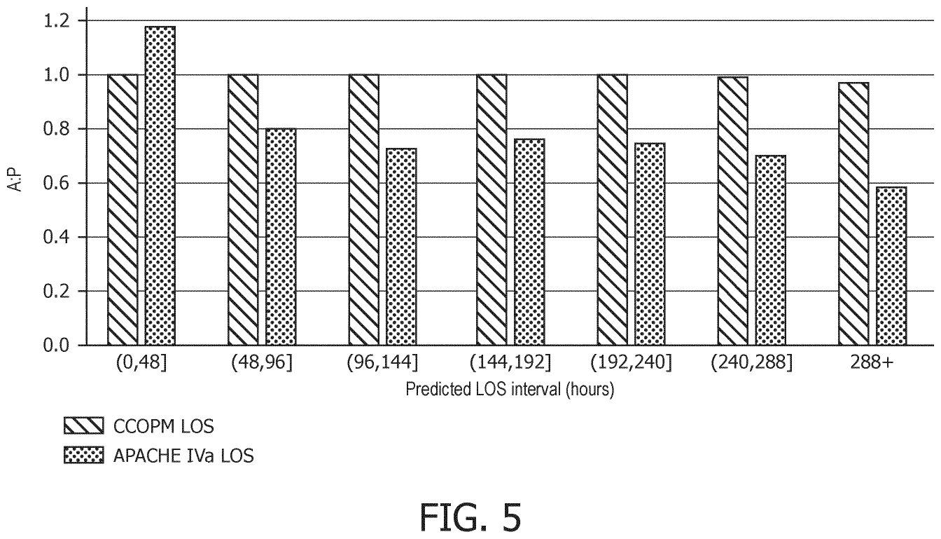

With reference to FIG. 4A, FIG. 4B, and FIG. 4C, the inventive ICU LOS model according to another example is compared with the APACHE IVa model over a dataset spanning from 2004 to 2013. In particular, an actual to predicted ratio (A:P) of the LOS averages, the R² values, and the median absolute error (MedAE) are illustrated for these models. As shown, the inventive ICU LOS model (labeled “CCOPM LOS”) presents an A:P ratio closer to 1, a smaller MedAE, and a higher R² value when compared to APACHE IVa for this period. The inventive ICU LOS model also presented good calibration, having stable A:P ratios for all ranges of predicted LOS within the period, as shown in FIG. 5.

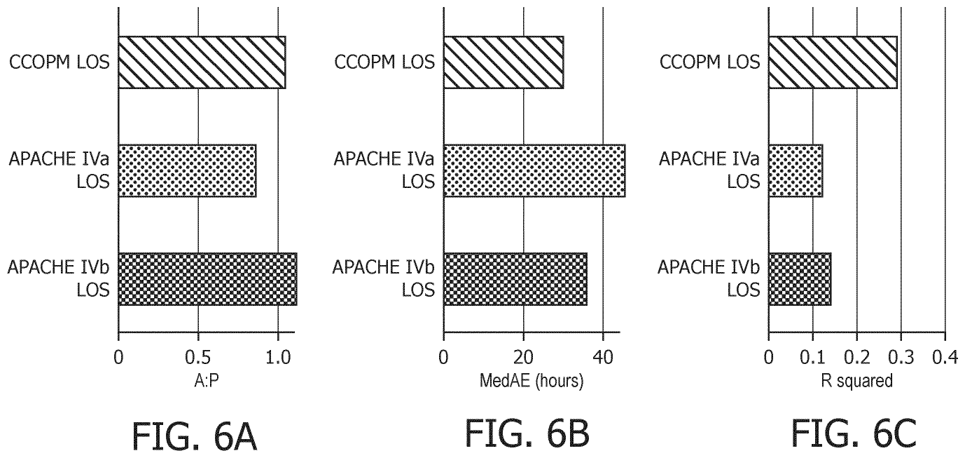

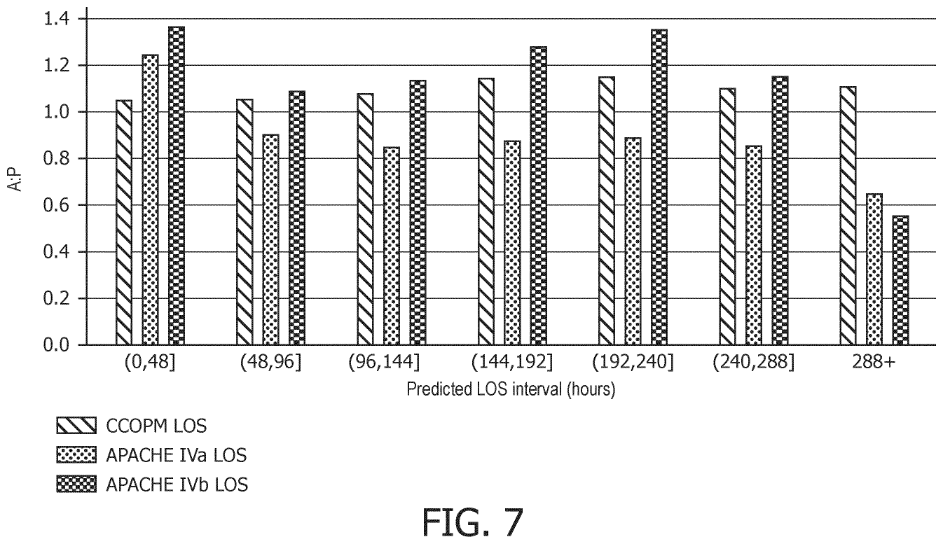

Similarly, with reference to FIG. 6A, FIG. 6B, and FIG. 6C, the inventive ICU LOS model according to another example is compared with the APACHE IVa and APACHE IVb models over a dataset spanning from 2020 to 2021. As shown, the inventive ICU LOS model presents an A:P closer to 1 than the comparative models, a smaller MedAE, and a higher R². Further, as shown in FIG. 7, the inventive ICU LOS model presents A:P values closer to 1 and that are more stable throughout different LOS ranges relative to the comparative models.

According to an embodiment, the ICU LOS prediction system is configured to process many thousands or millions of datapoints to extract the plurality of different defined ICU LOS prediction features, to generate the prediction of ICU LOS for the patient, and to display the prediction of ICU LOS for the patient to a user via the user interface. Further, preferably data for 100s or 1000s of patients are used to train the ICU LOS prediction model 264. Accordingly, the ICU LOS prediction system is configured to process millions of datapoints to extract the plurality of different defined ICU LOS prediction features for these 100s or 1000s of patients and use that data to train the ICU LOS prediction model 264. This requires millions or billions of calculations, which a human mind could not perform in a lifetime. Further, since training the ICU LOS prediction model 264 utilizes a unique data set, the stored trained ICU LOS prediction model is a novel model.

By providing improved prediction of the likelihood of ICU LOS for a patient, this novel ICU LOS prediction system has an enormous positive effect on patient care compared to prior art systems. Improved understanding of the length of stay of a patient in the ICU LOS can improve patient care and health, thereby saving lives not only in the ICU but in the entire care facility.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for predicting a length of stay (LOS) for a patient in an intensive care unit (ICU), the method comprising:
obtaining, from an electronic medical records database, a plurality of records for the patient in the ICU covering at least a first time period;
extracting, from the obtained plurality of records, a plurality of different defined ICU LOS prediction features for the patient;
analyzing the extracted plurality of different defined ICU LOS prediction features using a trained ICU LOS prediction model; and
predicting by the trained ICU LOS prediction model, an ICU LOS for the patient, wherein the predicted ICU LOS comprises a predicted probability of the patient leaving the ICU at a plurality of different timepoints.

2. The method of claim 1, wherein the first time period is at least 24 hours.

3. The method of claim 1, wherein the extracted plurality of different defined ICU LOS prediction features for the patient comprises one or more of ICU length of stay, ICU discharge status deceased, age, sex, pre-ICU admission lead time, elective surgery, ventilated, artificial airway, BMI, mean blood pressure, diastolic blood pressure, systolic blood pressure, heart rate, oxygen saturation, respiratory rate, PaCO2, glucose, lactate, pH, while blood cell count, hemoglobin, albumin, sodium, potassium, creatinine, GCS total, ICU type, ICU admission source, or diagnostic group.

4. The method of claim 1, wherein the predicting is performed by an ICU LOS prediction system that is a component of a patient data management systems (PDMS), a patient flow management system, or a patient monitoring system.

5. The method of claim 1, wherein the patient is a historical patient.

6. The method of claim 1, wherein the trained ICU LOS prediction model is configured to analyze the extracted plurality of different defined ICU LOS prediction features and predict the ICU LOS for the patient when one or more not required ICU LOS prediction features are missing from the obtained plurality of records.

7. (canceled)

8. The method of claim 16, wherein at least some of the extracted plurality of different health features are binned in bins prior to training the ICU LOS prediction model, and wherein the bins comprise a bin comprising missing data.

9. The method of claim 1, wherein ICU LOS prediction model comprises time-to-event analysis with competing risks, the time-to-event comprising time elapsed from admission of the patient to the ICU until a time the patient leaves the ICU, and the competing risks comprising an exit status of the patient either alive or deceased.

10. An intensive care unit (ICU) length of stay (LOS) prediction system for predicting an LOS for a patient in an ICU, the system comprising:
an electronic medical records database comprising a plurality of records for a plurality of patients; and
a processor configured to:
(i) obtain, from the electronic medical records database, a plurality of records for the patient in the ICU covering at least a first time period;
(ii) extract, from the obtained plurality of records, a plurality of different defined ICU LOS prediction features for the patient;
(iii) analyze the extracted plurality of different defined ICU LOS prediction features using a trained ICU LOS prediction model; and
(iv) predict, by the trained ICU LOS prediction model, an ICU LOS for the patient, wherein the predicted ICU LOS comprises a predicted probability of the patient leaving the ICU at a plurality of different timepoints.

11. The system of claim 10, wherein the extracted plurality of different defined ICU LOS prediction features for the patient comprises one or more of ICU length of stay, ICU discharge status deceased, age, sex, pre-ICU admission lead time, elective surgery, ventilated, artificial airway, BMI, mean blood pressure, diastolic blood pressure, systolic blood pressure, heart rate, oxygen saturation, respiratory rate, PaCO2, glucose, lactate, pH, while blood cell count, hemoglobin, albumin, sodium, potassium, creatinine, GCS total, ICU type, ICU admission source, or diagnostic group.

12. The system of claim 10, wherein the first time period is at least 24 hours.

13. The system of claim 10, wherein the ICU LOS prediction system is a component of a patient data management systems (PDMS) or a patient monitoring system.

14. (canceled)

15. The system of claim 10, wherein ICU LOS prediction model comprises time-to-event analysis with competing risks, the time-to-event comprising time elapsed from admission of the patient to the ICU until a time the patient leaves the ICU, and the competing risks comprising an exit status of the patient either alive or deceased.

16. The method of claim 1, wherein the ICU LOS prediction model is trained by:
obtaining, from an electronic medical records database, a plurality of historical records for each of a plurality of historical patients in an ICU;
extracting, from the obtained plurality of records, a plurality of different health features for each of the plurality of historical patients;
curating the extracted plurality of different health features to identify a plurality of different historical ICU LOS prediction features, wherein curating comprise identifying which of the different health features are required ICU LOS prediction features and which of the different health features are not required ICU LOS prediction features;
training the ICU LOS prediction model using the plurality of different historical ICU LOS prediction features; and
storing the trained ICU LOS prediction model.

17. The method of claim 1, further comprising:
presenting, via a user interface, the predicted ICU LOS for the patient.

18. The system of claim 10, wherein the ICU LOS prediction model is trained by:
obtaining, from an electronic medical records database, a plurality of historical records for each of a plurality of historical patients in an ICU;
extracting, from the obtained plurality of historical records, a plurality of different health features for each of the plurality of historical patients;
curating the extracted plurality of different health features to identify a plurality of different historical ICU LOS prediction features, wherein curating comprises identifying which of the different health features are required ICU LOS prediction features and which of the different health features are not required ICU LOS prediction features;
training the ICU LOS prediction model using the plurality of different historical ICU LOS prediction features; and
storing the trained ICU LOS prediction model.

19. The system of claim 10, further comprising:
a user interface configured to provide the predicted ICU LOS for the patient.

20. A non-transitory computer-readable storage medium having stored a computer program comprising instructions, which, when executed by a processor, cause the processor to:
obtain, from the electronic medical records database, a plurality of records for the patient in the ICU covering at least a first time period;
extract, from the obtained plurality of records, a plurality of different defined ICU LOS prediction features for the patient;
analyze the extracted plurality of different defined ICU LOS prediction features using a trained ICU LOS prediction model; and
predict, by the trained ICU LOS prediction model, an ICU LOS for the patient, wherein the predicted ICU LOS comprises a predicted probability of the patient leaving the ICU at a plurality of different timepoints.
